(19) United States
(12) Patent Application Publication
(10) Pub. No.: US 2005/0186635 A1
(43) Pub. Date: Aug. 25, 2005

(54) Electroactive surface-confinable molecules

(75) Inventors: Cynthia C. Bamdad, Boston, MA (US); Shoshana Bamdad Shendelman, New York, NY (US); Michael Frid, Medford, MA (US)

Correspondence Address: Timothy J. Oyer, Ph.D.;Wolf, Greenfield & Sacks, P.C., 600 Atlantic Avenue, Boston, MA, 02210-2206, US

(73) Assignee: Minerva Biotechnologies Corporation, Newton, MA (US)

(21) Appl. No.: 10/913,150

(22) Filed: Aug. 6, 2004

Related U.S. Application Data
(63) Continuation of application No. 10/683,862, filed on Oct. 9, 2003, now abandoned.
Application No. 10/683,862 is a continuation of application No. 10/304,462, filed on Nov. 26, 2002, now abandoned.
Application No. 10/304,462 is a continuation of application No. PCT/US01/40801, filed on May 25, 2001.
(60) Provisional application No. 60/277,861, filed on Mar. 22, 2001.
(60) Provisional application No. 60/207,387, filed on May 26, 2000.

(51) Int. Cl. G01N 33/53; G01N 33/567; C12Q 1/26; C07F 17/02

(52) U.S. Cl. 435/7.1; 556/143; 435/25

(57) ABSTRACT

The invention provides compositions, kits, methods, and species that include electroactive entities which can serve as signaling entities in chemical and/or biochemical assays. The electroactive species can be metallocenes, such as ferrocenes, including substituents that affect the oxidation/reduction potential (redox potential) of the species. By controlling the redox potential of the species, multiple species can be used in a single assay, each species having a different redox potential, for simultaneous signaling of different binding events. Additionally, species having redox potentials lower than 490 mV can be provided, allowing signaling within a potential range easily detectable in the presence of biological fluids.

### RELATED APPLICATIONS

This application is a continuation of U.S. patent application Ser. No. 10/683,862, filed Oct. 9, 2003, which is a continuation of U.S. patent application Ser. No. 10/304,462, filed Nov. 26, 2002, which is a continuation of PCT International Application No. PCT/US01/40801, filed May 25, 2001 which claims priority to U.S. application Ser. No. 60/227,861, filed Mar. 22, 2001, and to U.S. application Ser. No. 60/207,387, filed May 26, 2000.

### FIELD OF THE INVENTION

The present invention relates generally to chemical and biological assays and, more particularly, to a signaling molecule for detecting chemical or biological events, comprising a substituted metallocene.

### BACKGROUND OF THE INVENTION

Many areas of chemistry and biology involve determination of the existence and/or amount of a particular species (e.g. an analyte) in a fluid medium, within a solid sample, on a surface such as a cell surface, etc. Determining the existence or the amount of analyte is a well-developed field in many respects. For example, a variety of assays including competition assays, sandwich assays, and the like have been developed for detection of an analyte in a sample, a specific biological binding event, the activity of a candidate drug in drug screening, clinical diagnostics involving physiological analytes, etc.

Compounds containing redox active metals (typically, transition metals) can be used as signaling entities in various detection strategies. Moieties containing electroactive species such as redox-active metals eject electrons when they are subjected to their characteristic oxidation potentials, which is detectable electronically. Detection techniques such as Current Voltammetry (CV) and Alternating Current Voltammetry (ACV) measure current output as a function of voltage or potential and are used to quantitate oxidation and reduction of transition metals. In ACV, the voltage ramp contains an oscillating component such that each redox-active metal ejects many electrons (proportional to the frequency of the oscillating component) as the metals are repeatedly oxidized and reduced within a single scan.

In techniques such as CV and ACV, redox-active metals participating in an assay must be close to one electrode (a “working electrode”) of a multi-electrode electrochemical arrangement; the ability of ejected electrons to communicate with the working electrode is extremely distance dependent. In typical detection strategies, one binding partner is attached or drawn to the working electrode and a putative binding partner is labeled with a moiety containing a redox-active metal. If binding between the binding partner and the putative binding partner occurs, the transition-metal-labeled binding partner is recruited to the working electrode; ejected electrons are then within some critical tunneling distance and can transduce a detectable current output.

U.S. Pat. No. 5,223,117 (Wrighton et al.) describes self-assembly of a chemically-insensitive redox material, such as ferrocenyl thiol, and a chemically sensitive redox material, such as a quinone thiol, onto microelectrodes forming the basis for a two-terminal voltammetric microsensor, having reference and sensor functions on one electrode. Detection is accomplished by measuring a potential difference associated with current peaks for oxidation (or reduction) of the chemically insensitive and chemically sensitive redox materials, respectively, upon interaction of an analyte with the chemically sensitive redox material.

Various studies have addressed the issue of conductivity of “molecular wires” (see, for example, Chemical &Engineering News, Mar. 25, 1996, pg. 7). One reported study (Creager, et al. J. Am. Chem. Soc., 1999, 121, 1059-1064) involved the attachment of compound 1 and related compounds to a gold surface and reporting of electron-transfer 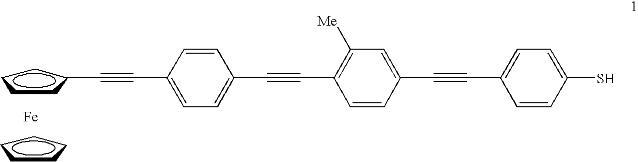
rates and electronic coupling factors for the attached ferrocene groups.

While signaling entities using redox active metals are known, improved detection strategies and redox active metals with improved oxidation characteristics, especially in connection with certain biological systems, would be advantageous.

### SUMMARY OF THE INVENTION

The present invention provides a series of compositions, kits, methods, and species associated with electroactive signaling entities. Various aspects of the invention can be used for a variety of purposes, for example, they can facilitate a variety of chemical or biological assay techniques. One aspect of the invention provides compositions. In one embodiment, a composition is provided comprising a substituted metallocene. Substituted metallocenes of the invention can include essentially any number of substituents on aromatic rings of the metallocene molecule.

In another embodiment, a composition is provided that includes a molecular species able to integrate into a self-assembled monolayer. The species comprises a metallocene including at least one, and preferably at least two substituents.

In another embodiment a composition of the invention includes a molecular moiety that promotes electron flow through a self-assembled monolayer connected to a metallocene including at least one, and preferably at least two substituents.

In another embodiment a composition is provided that includes a molecular moiety that promotes electron flow through a self-assembled monolayer connected to an electroactive species having a redox potential less than 490 mV.

The invention also provides a molecular species having a formula X—R—Y. X comprises a functional group selected to adhere to a surface and Y comprises an electroactive signaling entity having a redox potential of less than 490 mV or a metallocene including at least one, and preferably at least two substituents. R is a chemical bond, a spacer moiety that can form part of a self-assembled monolayer, a moiety that promotes electron flow through a self-monolayer, or a combination.

In another aspect the invention provides kits. One kit includes a first electroactive species having a first redox potential, immobilizable with respect to a first chemical or biological binding partner. A second electroactive species also is included in the kit, having a second redox potential. The second species is immobilizable with respect to a second chemical or biological binding partner.

In another aspect the invention provides a series of methods. One method involves determining a chemical or biological binding event indicated by a first redox potential of a first signaling entity in an assay involving also a second signaling entity having a second redox potential.

In another embodiment a method is provided that involves determining effectiveness of a candidate drug in inhibiting binding of a first binding partner to a second binding partner. The first binding partner, linked to a metallocene, is exposed to the second binding partner in the presence of a candidate drug. Binding of the first binding partner to the second binding partner is determined by determination of redox potential of the metallocene.

In another embodiment a method of the invention involves determining effectiveness of a candidate drug in inhibiting binding of a first binding partner to a second binding partner by exposing the first binding partner, immobilized with respect to a first signaling entity, and the candidate drug, immobilized with respect to a second signaling entity, to the second binding partner. Relative binding of the first binding partner or candidate drug to the second binding partner is determined by determining proximity of the first or second signaling entity to the second binding partner.

Other advantages, novel features, and objects of the invention will become apparent from the following detailed description of the invention when considered in conjunction with the accompanying drawings, which are schematic and which are not intended to be drawn to scale. In the figures, each identical or nearly identical component that is illustrated in various figures is represented by a single numeral. For purposes of clarity, not every component is labeled in every figure, nor is every component of each embodiment of the invention shown where illustration is not necessary to allow those of ordinary skill in the art to understand the invention.

### BRIEF DESCRIPTION OF THE DRAWINGS

FIG. 1 is a schematic illustration of one kit of the invention including electroactive signaling entities and binding partners fastened to colloid particles;

FIG. 2 schematically illustrates the kit of FIG. 1 interacting with a cell including different receptors on its surface; and

FIG. 3 illustrates schematically an arrangement for an assay for determining effectiveness of a candidate drug in inhibiting binding of a binding partner to a receptor.

### DETAILED DESCRIPTION OF THE INVENTION

Definitions

As used herein, the following definitions apply.

“Self-assembled monolayers” (SAMS) suitable for use in the present invention, and surfaces upon which they can be formed, are known, as described in U.S. Pat. No. 5,512,131 and International Patent Publication No. WO 96/29629, both of which are incorporated herein by reference. Surfaces and functional groups that will adhere to surfaces for self-assembled monolayer formation include, without limitation, gold, silver, copper, cadmium, zinc, palladium, platinum, mercury, lead, iron, chromium, manganese, tungsten, and any alloys of the above with sulfur-containing functional groups such as thiols, sulfides, disulfides, and the like; doped or undoped silicon with silanes and chlorosilanes; metal oxides such as silica, alumina, quartz, glass, and the like with carboxylic acids; platinum and palladium with nitrites and isonitriles; and copper with hydroxamic acids. Additional suitable functional groups include acid chlorides, anhydrides, sulfonyl groups, phosphoryl groups, hydroxyl groups and amino acid groups. Additional surface materials include germanium, gallium, arsenic, and gallium arsenide. Additionally, epoxy compounds, polysulfone compounds, plastics and other polymers may find use as surfaces to which functional groups can adhere in the invention. Additional materials and functional groups suitable for use in the invention can be found in U.S. Pat. No. 5,079,600, incorporated herein by reference. Self-assembled monolayers typically include linear molecules that facilitate close, aligned packing at a surface, such as omega-functionalized thiols, that is, those having the generalized structure R′—A—R″, where R′═—SH, A═—(CH₂)ₙ— where n=1-30, preferably 1-20, and R”═—CH₃; —OH; —O(CH₂)ₙH, where n=1-15, preferably 1-7; —CONH(CH₂)ₙH, where n=1-15, preferably 1-7; —NHCO(CH₂)ₙH, where n=1-15, preferably 1-7; —(OCH₂CH₂)ₙH, where n=1-15, preferably 1-7; and —COOH.

A “self-assembled monolayer-forming species” comprises a species that, when exposed to an appropriate surface with other, like species, e.g. provided with like species in an appropriate solution and exposed to an appropriate surface, will spontaneously form a self-assembled monolayer on the surface.

A species “able to integrate into a self-assembled monolayer” (which can be a self-assembled monolayer forming species) is a species having a chemical functionality favoring participation in a self-assembled monolayer comprising the species and other, self-assembled monolayer-forming species with which it is not chemically incompatible. For example, the species may include a functional group selected to adhere to a surface on which the self-assembled monolayer is formed, and may include a remainder portion that may be approximately linear (not highly-branched), but which does not facilitate close packing. Molecules including a significant amount of unsaturation, for example a series of interconnected aromatic rings, are examples. Such a species may or may not be a self-assembled monolayer-forming species. Typically, species that are able to integrate into a self-assembled monolayer but are not able themselves to form a self-assembled monolayer will be able to participate in formation of and integrate into a self-assembled monolayer when present in an amount of up to about 50% as a percentage of overall species including chemically-compatible self-assembled monolayer-forming species.

A species or moiety “that promotes electron flow through a self-assembled monolayer” (which can be a self-assembled monolayer forming species) will enhance the ability for an electron encountering a SAM-coated electrode to communicate with the electrode. In some cases this can involve allowing a fluid encountering the SAM-coated electrode to communicate electrically with the electrode. These species typically, because of their bulk or other conformation (which may be specifically designed into the molecule), create defects in an otherwise relatively tightly-packed SAM to prevent the SAM from tightly sealing the surface against fluids to which it is exposed; they cause disruption of the tightly-packed self-assembled structure, thereby defining defects that allow fluid to which the surface is exposed to communicate electrically with the surface. In this context, the fluid communicates electrically with the surface by contacting the surface or coming in close enough proximity to the surface that electronic communication via tunneling, or the like, can occur. A species or moiety “that promotes flow through a self-assembled monolayer” also includes by definition electrically-conductive molecules or electrically-conductive portions of molecules. Included also, by definition, are “molecular wires,” as described in the art. A non-limiting list of moieties that promote electron flow through a self-assembled monolayer includes 2-mercaptopyridine, 2-mercaptobenzothiazole, dithiothreitol, 1,2-benzenedithiol, 1,2-benzenedimethanethiol, benzene-ethanethiol, 2-mercaptoethylether, poly (ethynylphenyl thiol) (i.e. C₁₆H₁₀S).:

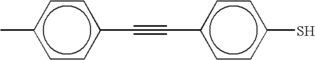

The term “binding partner” refers to a molecule that undergoes preferential binding with another molecule, the molecules defining a corresponding pair of molecules that exhibit mutual affinity or binding capacity, typically specific or non-specific binding or interaction, including biochemical, physiological, and/or pharmaceutical interactions, typified by biological binding pairs. The term “biological binding” refers to the interaction between a corresponding pair of biological molecules that exhibit specific or non-specific mutual affinity or binding capacity. Biological binding defines a type of interaction that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, drug candidates which can be synthetic or natural products as well as individual components, building blocks, or precursors of these, etc. For example, Protein A is a binding partner of the biological molecule IgG, and vice versa.

“Small molecule” means a molecule less than 5 kiloDalton, more typically less than 1 kiloDalton.

The term “candidate drug” refers to any medicinal substance used in humans or other animals. Encompassed within this definition are compound analogs, naturally occurring, synthetic and recombinant pharmaceuticals, hormones, antimicrobials, neurotransmitters, etc. This includes any substance (whether naturally occurring, synthetic or recombinant) which is to be evaluated for use as a drug for treatment of essentially any medical condition, such as disease, or prevention thereof. Evaluation typically takes place through activity in an assay, such as the screening assays of the present invention.

A variety of types of particles can be used in the invention. For example, “fluid suspendable particle” means a particle that can be made to stay in suspension in a fluid in which it is used for purposes of the invention (typically an aqueous solution) by itself, or can be maintained in solution by application of a magnetic field, an electromagnetic field, agitation such as stirring, shaking, vibrating, sonicating, centrifuging, vortexing, or the like. A “magnetically suspendable” particle is one that can be maintained in suspension in a fluid via application of a magnetic field. An electromagnetically-suspendable particle is one that can be maintained in suspension in a fluid by application of an electromagnetic field (e.g., a particle carrying a charge, or a particle modified to carry a charge). A “self-suspendable particle” is a particle that is of low enough size nd/or mass that it will remain in suspension in a fluid in which it is used (typically an aqueous solution) for at least 1 hour. Other self-suspendable particles will remain in suspension, without assistance, for 5 hours, 1 day, 1 week, or even 1 month, in accordance with the invention.

“Colloid,” as used herein, means nanoparticles, i.e. very small, self-suspendable or fluid-suspendable particles including those made of any material that is, e.g., inorganic or organic, polymeric, ceramic, semiconductor, metallic (e.g. gold), non-metallic, crystalline, amorphous, or a combination. Typically, colloid particles used in accordance with the invention are of less than 250 nm maximum dimension (cross section, edge, or any other dimension), more typically less than 100 nm, and in most cases are of about 2-30 nm maximum cross section. One class of colloids suitable for use in the invention is 10-30 nm in maximum cross section, and another about 2-10 nm in maximum cross section. As used herein this term includes the definition commonly used in the field of biochemistry. One type of colloid particle is a semiconductor nanocrystal, which is a particle of matter, typically with a maximum dimension of less than 100 or 75 nanometers, and more typically less than 50 or 25 nanometers. A semiconductor nanocrystal may have a Radius that is smaller than the bulk exciton Bohr radius. In a semiconductor nanocrystal, the addition or removal of an electron may change or alter its electronic properties. In certain semiconductor nanocrystals, these electronic properties may include fluorescence, or emission, of light, such as visible light, infrared light, ultraviolet light, or radio frequency radiation.

A “moiety that can coordinate a metal” means any molecule that can occupy at least two coordination sites on a metal atom, such as a metal binding tag or a chelate.

A “chelate coordinating a metal” or “metal coordinated by a chelate”, refers to a metal coordinated by a chelating agent that does not fill all available coordination sites on the metal, leaving at least two coordination sites available for binding via a metal binding tag. Examples of suitable chelates include nitrilotriacetic acid, 2,2′-bis(salicylideneamino)-6,6′-demethyldiphenyl, or 1,8-bis(a-pyridyl)-3,6-dithiaoctane, which can be used with a hexacoordinate metal such as nickel.

A “metal binding tag” refers to a moiety such as a group of molecules that can coordinate, i.e. become fastened to, a metal that is also coordinated by a chelate. Suitable groups of such molecules include amino acid sequences including, but not limited to, histidines and cysteines (“polyamino acid tags”, exemplified by “histidine tags”). Polyamino acid tags can include two to ten amino acid residues, such as histidine residues, which can be readily positioned at either the amino- or carboxy-terminus of a peptide or protein. A polyamino acid tag of six to ten residues is preferred.

A “metal binding tag/metal/chelate linkage” defines a linkage between two or more moieties in which one is fastened to a metal binding tag, another is fastened to a chelate coordinating a metal, and the binding tag binds the metal.

“Signaling entity” means an entity that is capable of indicating its existence in a particular sample or at a particular location.

“Immobilizable” as used herein, means provided with chemical or biological functionality allowing immobilization, or being immobilized to.

As used herein, a component that is “immobilized relative to” another component either is fastened to the other component or is indirectly fastened to the other component, e.g., by being fastened to a third component to which the other component also is fastened, or otherwise is transitionally associated with the other component. For example, a signaling entity is immobilized with respect to a binding species if the signaling entity is fastened to the binding species, is fastened to a colloid particle to which the binding species is fastened, is fastened to a dendrimer or polymer to which the binding species is fastened, etc. A colloid particle is immobilized relative to another colloid particle if a species fastened to the surface of the first colloid particle attaches to an entity, and a species on the surface of the second colloid particle attaches to the same entity, where the entity can be a single entity, a complex entity of multiple species, a cell, another particle, etc.

As used herein, “fastened to or adapted to be fastened”, in the context of a species relative to another species or to a surface of an article, means that the species is chemically or biochemically linked via covalent attachment, attachment via specific biological binding (e.g., biotin/streptavidin), coordinative bonding such as chelate/metal binding, or the like. For example, “fastened” in this context includes multiple chemical linkages, multiple chemical/biological linkages, etc., including, but not limited to, a binding species such as a peptide grown on a polystyrene bead, a binding species specifically biologically coupled to an antibody which is non-specifically biologically bound to a protein such as protein A, which is covalently attached to a bead, a binding species that forms a part (via genetic engineering) of a molecule such as GST or Phage, which in turn is specifically biologically bound to a binding partner covalently fastened to a surface (e.g., glutathione in the case of GST), etc. As another example, a moiety covalently linked to a thiol is adapted to be fastened to a gold surface since thiols bind gold very strongly, perhaps covalently. Similarly, a species carrying a metal binding tag is adapted to be fastened to a surface that carries a molecule covalently attached to the surface (such as thiol/gold binding) which molecule also presents a cheiate coordinating a metal. A species also is adapted to be fastened to a surface if it carries a particular nucleotide sequence, and the surface includes a complementary nucleotide sequence. The definition of “fastened” includes some non-specific binding, namely, intentional adherence of a species to a surface for purposes of a technique of the invention. Non-specific binding falling under the definition of “fastened” in the invention will result in attachment that withstands routine washing steps associated with assays of the invention. Specifically, a non-specifically bound species is “fastened” to a surface if it will withstand at least one routine washing with an aqueous wash solution and a buffer, such as a phosphate buffer for maintaining the fluid at physiological pH.

“Specifically fastened” or “adapted to be specifically fastened” means a species is chemically or biochemically linked to a surface as described above with respect to the definition of “fastened to or adapted to be fastened”, but excluding all non-specific binding.

“Non-specific binding”, as used herein, is given its ordinary meaning in the field of biochemistry.

The term “sample” refers to any cell, tissue, or fluid from a biological source (a “biological sample”, or any other medium that can advantageously be evaluated in accordance with the invention including, but not limited to, a biological sample drawn from a human patient, a sample drawn from an animal, a sample drawn from food designed for human consumption, a sample including food designed for animal consumption such as livestock feed, an organ donation sample, a synthetic sample, a sample drawn from a natural product or the like.

A “sample suspected of containing” a particular component means a sample with respect to which the content of the component is unknown. For example, a fluid sample from a human suspected of having a particular physiological condition such as disease, but not known to have the condition, defines a sample suspected of containing physiological species characteristic of the condition. “Sample” in this context includes naturally-occurring samples, such as physiological samples from humans or other animals, samples from food, livestock feed, etc., as well as “structurally predetermined samples”, which are defined herein to mean samples, the chemical or biological sequence or structure of which is a predetermined structure used in an assay designed to test whether the structure is associated with the condition. For example, a “structurally predetermined sample” includes a peptide sequence, random peptide sequence in a phage display library, a structurally-predetermined synthetic sample, a structurally-predetermined sample from a natural product, and the like. Typical samples taken from animals include blood, urine, ocular fluid, saliva, cerebro-spinal fluid, fluid or other samples from tonsils, lymph nodes, needle biopsies, etc.

“Determining” means determining the existence of, i.e. detecting, or determining the amount or relative amount of, i.e., qualitatively assessing, a species. For example, qualitative determining can involve detecting whether a particular electroactive signaling entity is proximate a working electrode using CV or ACV or charge-counting apparatus in a conventional manner, and quantitative determining can involve determining the amount of an electroactive signaling entity proximate an electrode by measuring oxidation or reduction current of the species proximate the electrode as compared to current resulting from oxidation and/or reduction of a second species proximate the same or a different electrode, the quantity of which is known, for example.

The term “coordination”, or “coordinated” refers to an interaction in which one multi-electron pair donor, such as a chelating agent or a polyamino acid tag coordinatively bonds (is “coordinated”), to a metal with a degree of stability great enough that an interaction that relies upon such coordination for detection in an assay can be determined.

The term “coordination site” refers to a point on a metal that can accept an electron pair donated, for example, by a chelating agent.

The term “free coordination site” refers to a coordination site one a metal that is occupied by a water molecule or other species that is weakly donating relative to a polyamino acid tag.

The term “coordination number” refers to the number of coordination sites on a metal that are available for accepting an electron pair.

The present invention provides novel electroactive species and kits which can be used in biological and/or chemical assays, as well as a variety of assays which can be conducted with, or independently of, the novel species. It will be apparent to those of ordinary skill in the art that many of the species, kits, and methods of the invention described below would be used in connection with electrochemical apparatus such as CV and ACV apparatus able to apply a varying voltage to an electroactive species and to measure resulting oxidation and/or reduction of the species. Such apparatus is well-known to those of ordinary skill in the art and will not be described in detail herein.

One aspect of the invention involves carrying out assays involving electroactive species serving as electroactive detection reagents (electroactive signaling entities) in biological samples containing naturally-occurring redox-active species that would complicate detection using conventional electroactive reagents. Specifically, assays can be conducted in connection with biologically-derived test solutions containing substances such as hemes, flavins, urea, redox-active co-enzymes (vitamins), natural anti-oxidants and the like, that are also redox-active. The presence of these species generate a high background current that can mask conventional label-specific oxidation peaks. Specifically, nature has eliminated the risk of random oxidation of these naturally occurring redox-active species by background potential fields within the body by making these species oxidize at potentials over about 450 mV. Unfortunately, this region (over 450 mV) is an area where many conventional redox-active detection reagents also oxidize, thus detection using conventional redox-active detection reagents can be complicated by oxidation of naturally-occurring species.

Moreover, the characteristic oxidation potential of many conventional redox-active signaling agents can be shifted to higher potentials when they are placed in different biochemical environments. Certain binding events can cause a redox-active metal signaling complex, attached to a binding ligand, to become aggregated with other proteins, shielding the metal from an electric field applied in a detection step. This can result in an apparent shift of the complex's characteristic oxidation potential. For example, it is not uncommon for a ferrocene complexed with a characteristic oxidation potential of 450 mV to shift to an oxidation potential of 800-900 mV when bound to certain protein targets. Additionally, many other substances used in typical CV or ACV assays (including gold) oxidize between 800-900 mV, which can generate background current peaks that complicate interpretation of results.

Accordingly, one aspect of the invention involves a series of species including features that make them particularly suited for biological assays. In one embodiment, electroactive species suitable for use as signaling entities in biological assays are provided that oxidize at relatively low potentials, specifically, at potentials low enough such that they provide signaling capability via oxidation or reduction that is not complicated by the redox potentials of common physiological substances. One such electroactive species of the invention has a redox potential of less than 490 mV (vs. Ag AgCl). In a preferred embodiment, the electroactive species has a redox potential of less than about 400 mV, preferably less than about 350 mV, more preferably less than about 300 mV, and more preferably still less than about 250 mV.

Electroactive signaling entities of the invention, and other embodiments of the invention involving species and kits, can be made in certain embodiments to be able to able to integrate into self-assembled monolayers, making them particularly useful in connection with assays involving attachment to a surface such as an electrode surface, colloid surface, or the like. In preferred embodiments of the invention involving self-assembled monolayers, a gold surface, such as a layer of gold deposited on a glass substrate, a gold electrode, or a surface of a gold colloid, or the like is used, with a species forming or participating in the self-assembled monolayer (such as an electroactive species) including or attached to a thiol (which binds to gold).

Certain embodiments of the invention make use of self-assembled monolayers (SAMs) on surfaces, such as surfaces of colloid particles, and articles such as colloid particles having surfaces coated with SAMs. In one set of preferred embodiments, SAMs formed completely of synthetic molecules completely cover a surface or a region of a surface, e.g. completely cover the surface of a colloid particle. “Synthetic molecule”, in this context, means a molecule that is not naturally occurring, rather, one synthesized under the direction of human or human-created or human-directed control.

“Completely cover” in this context, means that there is no portion of the surface or region that directly contacts a protein, antibody, or other species that prevents complete, direct coverage with the SAM. I.e. in preferred embodiments the surface or region includes, across its entirety, a SAM consisting completely of non-naturally-occurring molecules (i.e. synthetic molecules). The SAM can be made up completely of SAM-forming species that form close-packed SAMs at surfaces, or these species in combination with molecular wires or other species able to promote electronic communication through the SAM (including defect-promoting species able to participate in a SAM), or other species able to participate in a SAM, and any combination of these. Preferably, all of the species that participate in the SAM include a functionality that binds, optionally covalently, to the surface, such as a thiol which will bind to a gold surface covalently. A self-assembled monolayer on a surface, in accordance with the invention, can be comprised of a mixture of species (e.g. thiol species when gold is the surface) that can present (expose) essentially any chemical or biological functionality. For example, they can include tri-ethylene glycol-terminated species (e.g. tri-ethylene glycol-terminated thiols) to resist non-specific adsorption, and other species (e.g. thiols) terminating in a binding partner of an affinity tag, e.g. terminating in a chelate that can coordinate a metal such as nitrilotriacetic acid which, when in complex with nickel atoms, captures histidine-tagged binding species. The present invention provides a method for rigorously controlling the concentration of essentially any chemical or biological species presented on a colloid surface, for example electroactive surface-confinable molecules, chelates, binding partners, etc. In many embodiments of the invention the self-assembled monolayer is formed on gold colloid particles.

The invention also provides a series of methods, including assays utilizing at least one, preferably at least two electroactive species having different redox potentials for purposes that will become more apparent from the description below. The two redox potentials can be less than or greater than 490 mV. In preferred embodiments, at least two electroactive species each have a redox below 490 mV or other, preferred redox potentials listed herein.

In an aspect of the invention where at least two electroactive species of different redox potential are used, the electroactive species can be selected among any of a wide variety of species known to, and readily selectable by, those of ordinary skill in the art. Preferred electroactive species include redox active molecules including transition metal complexes, i.e., complexes including metals selected from, but not limited to, cadmium, copper, cobalt, palladium, zinc, iron, ruthenium, rhodium, osmium, rhenium, platinum, scandium, titanium, vanadium, chromium, manganese, nickel, molybdenum, technetium, tungsten, and iridium. Particularly preferred are ruthenium, osmium, iron, platinum, and palladium, with ruthenium and iron being especially preferred. Complexes including these metals will include the metals along with ligands such as, for example, isonicotinamide, imidazole, bipyridine, terpyridine, phenanthrolines, carbon monoxide, isocyanide, and metallocene ligands, including substituted derivatives of the above. Other ligands will be apparent to those of ordinary skill in the art. A particularly preferred metal complex of the invention is a metallocene complex, especially ferrocene, which includes an iron atom and two cyclopentadiene ligands.

A particularly preferred set of embodiments, according to the aspect of the invention involving electroactive species having redox potential less than 490 mV, involves metallocenes, particularly ferrocene, substituted with substituents on one or both cyclopentadiene rings that affect the electronic characteristic of the metallocene so as to shift its redox potential to less than 490 mV or other potentials mentioned herein. Suitable substituents include, without limitation, a halide such as bromine, —NR₂, —OR, —OCR₃, —NRCOR₃, —C₆R₅, carboxylates, and alkyl groups such as CR₃. R preferably is hydrogen or alkyl, most preferably hydrogen. As noted herein, a variety of substituents can be selected including different substituents on a single metallocene, to provide a particular redox characteristic for the metallocene. Those of ordinary skill in the art will understand how to select various substituents to achieve a desired redox potential. For example, methyl substituents will tend to drive the redox potential of the metallocene down, while carboxylates will tend to drive the redox potential up. The invention allows for tailoring the redox potential of a metallocene at any location through a range of from about −200 mV to about 800 mV vs. Ag/AgCl so that, for example, a variety of species can simultaneously be detected in parallel in a single electrochemical experiment, whether in a biological fluid or a non-biological fluid such as a buffer. Of course, in biological fluids it generally is advantageous to use electroactive species having a redox potential less than 490 mV, as described herein. In one preferred embodiment a metallocene such as ferrocene includes at least one, preferably at least two substitutes, preferably two electron-donating substituents, preferably methyl groups, for example one on each cyclopentadiene ring, i.e., a molecule of the formula Fe(C₅H₄Me)₂. Any number of additional substituents can be provided resulting, for example, in a electroactive species having a formula Fe(C₅Me₄H)₂. These substituents can be selected, for a particular metallocene, to all be electron-donating, all electron-accepting, or a combination. That is, in each embodiment described herein, any number of substituents can be provided and the substituents can be the same or different. In this manner, a metallocene can be tailored to have a particular redox potential by selecting a suitable set of substituents that provide an appropriate electronic characteristic within the molecule. This is useful for carrying out simultaneous, multiple detections as described herein. A metallocene including at least one, and preferably at least two substituents that forms part of a species able to integrate into a self-assembled monolayer defines another embodiment of the invention.

In one set of embodiments involving species and kits of the invention, a species including an electroactive moiety, such as one having a redox potential less than 490 mV or a substituted metallocene, is immobilized relative to or fastened to, or otherwise forms a part of a species able to integrate into a self-assembled monolayer, or a self-assembled monolayer-forming species, where the component that allows integration into the self-assembled monolayer or that promotes formation of the self-assembled monolayer is a saturated species such as an alkyl chain. In another set of embodiments, the species including the electroactive moiety includes also a moiety that promotes electron flow through a self-assembled monolayer. This facilitates better electronic communication between the electroactive entity and a surface (such as an electrode) upon which it resides, improving determination of the presence, quantity, and/or redox shift of the entity. In either of these cases, the moiety typically is able to integrate into a self-assembled monolayer, but may not be a self-assembled monolayer-forming species itself, thus it may be provided in a self-assembled monolayer in combination with a self-assembled monolayer-forming species.

In one set of embodiments, kits, methods, species, and reagents of the invention can include metallocenes that are substituted with any number of substituents including one, two, three, four, five, six, seven, eight, nine or ten substituents, where the metallocene can be, but need not be, immobilized with respect to or form part of a species able to integrate into a self-assembled monolayer or a self-assembled monolayer-forming species. That is, one aspect of the invention simply involves a metallocene with any number of substituents. In one set of embodiments the metallocene is ferrocene. In one embodiment, the metallocene is constructed so as to be immobilized with respect to a surface of an article, or to a polymer or dendrimer molecule, or to another molecule such as a biological molecule, especially any of the binding partners described above. Those of ordinary skill in the art are aware of a wide variety of chemical pathways for linking a metallocene to a polymer molecule, a polymeric surface, a dendrimer, or surfaces of a wide variety of articles. For example, metallocenes can be immobilized relative to a surface by way of a linker such as described above with respect to linking self-assembled monolayer-forming species to surfaces, or other linkers known in the art.

As specific examples, surfaces such as carboxy-terminated surfaces can be treated with EDC/NHS chemistry, and a metallocene carrying a linking entity such as an amine can fasten thereto. Similarly, metallocenes can be immobilized with respect to biological molecules such as proteins by treating carboxy groups of the protein with EDC/NHS chemistry and fastening the metallocene to the protein via an amine linker. The reverse also can be carried out, that is, a carboxy group can be presented by a metallocene and a surface can present an amine, or a protein can present an amine, and EDC/NHS chemistry can be used to link the metallocene to the surface or to the protein. Similarly, polymers or dendrimers can include carboxy groups or amines with amines or carboxy groups, respectively, on metallocenes, and EDC/NHS chemistry can be used to link the metallocene to the polymer or dendrimer. Additionally, electroactive signaling entities of the invention can be covalently attached to a BSA-coated gold surface such as a BSA-coated colloid.

This knowledge in the art can be used to implement this aspect of the invention which involves a substituted metallocene linked to a surface, a polymer, a dendrimer, or a biological molecule. The metallocene can be any metallocene disclosed herein, and can be used in any of the assays described herein. Where an electroactive signaling entity of the invention is immobilized with respect to a polymer or dendrimer, the polymer or dendrimer also can carry an immobilized probe molecule, i.e., a member of a biological binding partner pair.

Another aspect of the invention involves methods of determining the presence and/or amount of a chemical or biological species in a particular environment, which can make use of any of the species, compositions, and kits of the invention. Methods involving assays for the determination of binding between chemical or biological binding partners benefit especially from the present invention. In another aspect the invention provides kits useful for a variety of purposes, including but not limited to the methods of the invention described herein. Kits of the invention can include any one or a combination of species of the invention. For example, one kit of the invention includes two different electroactive species each immobilizable with respect to a different chemical or biological binding partner. The electroactive species can each have redox potentials below 490 mV, can be substituted metallocenes, can be linked to a species able to integrate into a self-assembled monolayer, can be linked to a species able to promote electron flow through a self-assembled monolayer, etc.

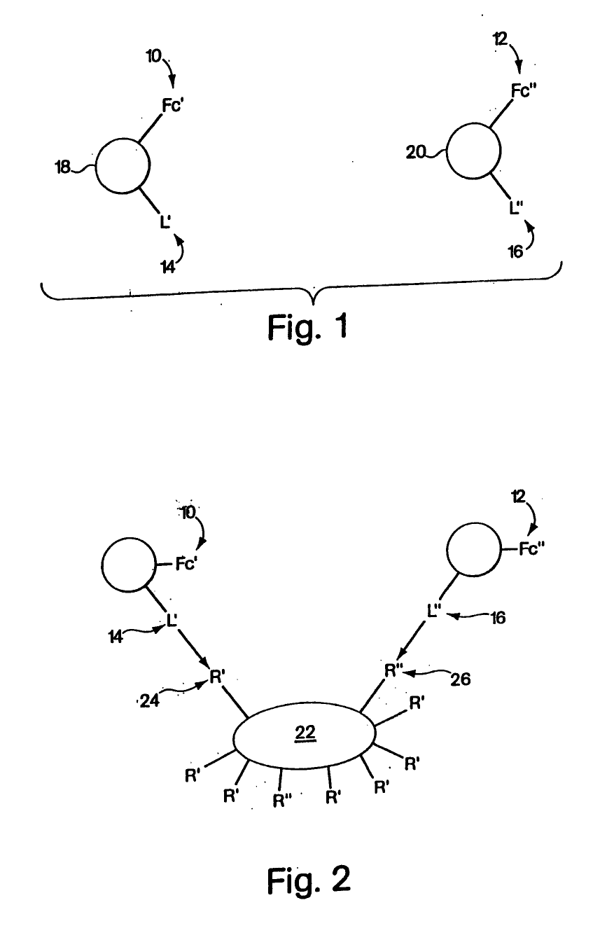

Referring now to FIG. 1, one kit of the invention includes a first electroactive species 10, exemplified by a first ferrocene (Fc′) and a second electroactive species 12, exemplified by a second ferrocene (Fc″). Each of the electroactive species 10 and 12 is immobilizable with respect to different chemical or biological binding partners, specifically, binding partners 14 and 16, respectively (exemplified as ligands L′ and L″).

In the arrangement of FIG. 1, the electroactive species are immobilized with respect to the binding partners by being immobilized to common colloid particles. Specifically, electroactive species 10 is linked to colloid particle 18 to which binding partner 14 also is linked, and electroactive species 12 is linked to colloid particle 20 to which binding partner 16 also is linked. The electroactive species and binding partners can be linked to the colloid particles in any of a variety of ways. In preferred embodiments each is linked to the colloid particle via a metal binding tag/metal/chelate linkage.

In an arrangement where a metal binding tag/metal/chelate linkage is used, a chelate can form part of a SAM on colloid particle 18 and/or 20. The chelate coordinates a metal, and a metal binding tag such as a polyamino acid tag can be incorporated into any or all of electroactive species 10 or 12, or binding partners 14 or 16, giving any or all of those species the ability to be immobilized to colloid particle 18 or 20 by coordination of the metal binding tag to the metal. Thus, an electroactive species arranged to be linked to either colloid particle can be represented by X—R—Y, where X is a functional group selected to adhere to the surface of the particle, R is a chemical bond, a spacer moiety that can form part of a self-assembled monolayer, a moiety that promotes electron flow through a self-assembled monolayer, or a combination, and Y comprises an electroactive signaling entity, which can be a low-redox entity such as a substituted metallocene. In this case R and Y can be connected to each other via a binding tag/metal/chelate linkage. In an alternate immobilization technique, any of species 10, 12, 14, or 16, most often species 14 or 16, can carry a terminal cysteine and fasten thereby to a gold surface of colloid particle 18 or 20.

The arrangement of FIG. 1 can define a kit including a plurality of colloid particles 18 carrying electroactive species 10 and binding partners 14, contained in a first package, and a plurality of colloid particles 20 carrying electroactive species 12 and binding partners 16, contained in a second package. Kits of the invention generally will include separate components in separate packages, each of the packages contained in a single, larger package optionally including instructions for use of the various packaged components. In one method in which the kit of FIG. 1 can be used, a plurality of colloid particles 18 and 20 are exposed to an analyte that will bind to one of binding partners 14 or 16, but not both. The analyte is allowed to bind to one of the binding partners, and the electroactive signaling entity 10 or 12 to which the analyte is thereby immobilized is detected, for example via recruitment of the analyte to a suitable electrode. For example, if the analyte can be simultaneously bound to an electrode-bound species, optionally presented as part of a self-assembled monolayer on the electrode, then the colloid particle immobilized with respect to the analyte can readily be recruited to the electrode where proximity of the signaling entity (e.g. Fc′ or Fc″) to the electrode can be detected. Alternatively, magnetic beads carrying species that will fasten to the analyte can be provided, followed by a magnetic recruitment of the beads to an electrode surface, drawing therewith the binding partner to which the analyte has bound along with the accompanying colloid particle and electroactive signaling entity. Alternatively, rather than being allowed to fasten to an analyte which then is recruited to a surface for detection, colloid particles 18 and 20 and accompanying electroactive entities and ligands can be exposed to a surface at which the analyte is bound, such as a cell carrying a receptor that defines the analyte. Thus, the arrangement of FIG. 1 facilitates a method of the invention that involves determining a chemical or biological binding event indicated by a first redox potential of a first signaling entity in an assay involving also a second signaling entity having a second redox potential.

In another embodiment utilizing the kit of FIG. 1, colloid particles 18 and 20 carrying electroactive signaling entities and ligands can be used to simultaneously perform two assays in a single sample solution.

The use of two distinct signaling entities 10 and 12, each immobilized with respect to a different binding partner, also allows for the comparison of one binding event to another. For example, and with reference to FIG. 2, a single cell 22 can be simultaneously bound by two binding partners (ligands) 14 and 16, where ligand 14 is selected to bind a receptor R″ (24) that is constitutively expressed, i.e., its concentration at the surface of cell 22 is fixed and is known, and binding partner 16 is selected to bind to a second cell receptor R″ (26) whose expression level at the surface of cell 22 is not known. When the system is provided in approximate proximity to an appropriate electrode (via arrangements and conditions known to those of ordinary skill in the art) the signal of electroactive species 10 can be used to calibrate, or normalize, the signal generated by electroactive species 12. As shown in FIG. 2, cell 22 includes significantly less of second receptor 26 (unknown level) than first receptor 24, and an amount of current flowing at the electrode during oxidation and/or reduction of electroactive signaling entity 12 will be proportionately lower to the amount of current flowing during oxidation and/or reduction of electroactive signaling entity 10. If the amount of receptor 24 is known, then the amount of receptor 26 at cell 22 can be determined by comparing these relative currents. If the amount of receptor 24 is unknown, but is invariant, then at least the relative amount of the receptors at the cell surface can be determined and, with the amount of receptor 24 invariant from cell to cell, then the concentration of receptor 24 of the cell can be determined absolutely by a series of comparative experiments in which at least one experiment involves a known amount of a receptor 26. Alternatively, the relative amounts of receptors 24 and 26 at the surface can lead to determination of the absolute amount of either receptor at the surface from other known criteria as would be apparent to those of ordinary skill in the art. This strategy eliminates signal variation that stems from a varying number of cells bound to a single electrode. The normalization of the signal also can be useful in cases where the expression level of second receptor 26 is examined in response to candidate therapeutic agents or drugs at a particular stage of cell cycle progression.

The arrangement shown in FIG. 2 also allows the derivation of information about how the presence of one receptor affects the expression of another receptor. The technique described above can provide this determination.

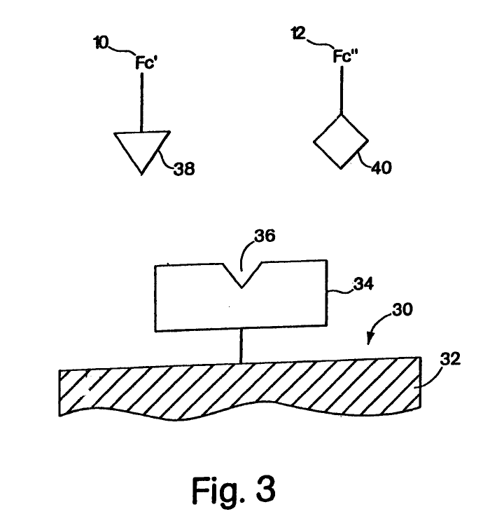

Another example of a method involving determining chemical or biological binding indicated by a first redox potential of a first signaling entity in an assay involving also a second signaling entity having a second redox potential is a competitive binding assay such as one shown in FIG. 3. In FIG. 3 a surface 30 of an electrode 32 (which can be present, but is not shown in FIGS. 1 and 2) carries an immobilized target receptor 34 having a binding site 36. Receptor 34 can be immobilized with respect to surface 30 by any convenient means. For example, it can form part of a self-assembled monolayer on surface 30 including a variety of self-assembled monolayer-forming species (not shown) and a species attached to receptor 34 that is able to integrate into or assist in formation of the self-assembled monolayer and that includes a functional group selected to adhere to species 30.

In FIG. 3 a first ligand 38 is immobilized with respect to electroactive signaling entity 10. Ligand 38 has the ability to biologically bind to receptor 34 at site 36. A candidate drug 40, potentially having the ability also to bind to site 36 of receptor 34, is immobilized with respect to electroactive signaling entity 12. Ligand 38 and candidate drug 40 can be immobilized with respect to entities 10 and 12, respectively, by a variety of arrangements including one in which each is linked to a colloid particle to which the signaling entity also is linked, as illustrated in FIGS. 1 and 2. If candidate drug 40 can bind to site 36 in competition with ligand 38, then when ligand 38 and candidate drug 40 each are allowed to bind to site 36, then this can be determined; not only will a loss in a current peak associated with oxidation and/or reduction of electroactive species 10 proximate electrode 32 be detected, but gain of a current signal resulting from the proximity of electroactive species 12 to the electrode will be detected. In this way, relative binding of ligand 38 or candidate drug 40 to site 36 can be determined by determining proximity of either the ligand or the candidate drug to the electrode, indicating proximity of the ligand or the drug to the receptor. In another embodiment (not shown) an arrangement as illustrated in FIG. 3 is provided except that candidate drug 40 is not bound to any signaling entity. In this arrangement a decrease in current peak associated with electroactive species 10 in proximity to electrode 32 is indicative of effectiveness of candidate drug 40 in binding to receptor 36, and this is useful in many situations. The arrangement illustrated in FIG. 3 includes the further advantage that any difficulty in quantitation of decrease in current peak of species 10 resulting from the presence of candidate drug 40 is overcome in that the relative growth of the current peak attributable to species 12 in comparison to a decrease in a peak associated with species 10 can be used to quantify the effectiveness of candidate drug 40 in competing with binding between ligand 38 and receptor 34.

Signal calibration can be performed also, in accordance with another embodiment of the invention, in which a known, predetermined number of a particular ligand is attached to an electrode, and the ligand's binding partner immobilized with respect to a signaling entity (via, for example, attachment to a common colloid particle), provided in excess, is exposed to the electrode. In this case all ligands will bind a binding partner and, with knowledge of the number of ligands present, the number of signaling entities present at the surface is known and the amount of current associated with oxidation and/or reduction of each signaling entity can thereby be determined. Alternatively, an electrode carrying excess ligand can be exposed to a known number of signaling entity-bound binding partners, each of which will bind to the surface, whereby the amount of current per signaling entity can be determined.

The function and advantage of these and other embodiments of the present invention will be more fully understood from the examples below. The following examples are intended to illustrate the benefits of the present invention, but do not exemplify the full scope of the invention.

### EXAMPLES

Under conditions of alternating current voltammetry (ACV), molecule 1 (prior art; comparative) oxidizes at 490 mV. Conditions for obtaining ACV results were as follows. Molecule 1, along with triethylene glycol-terminated thiols, were self-assembled onto a gold-coated glass slide from solution in dimethyl formamide. The gold coating served as a working electrode. Using a Ag/AgCl reference electrode and a platinum auxiliary electrode, and using a CH Instruments Model 630 electrochemical analyzer, a standard ACV was attained at an overpotential of 25 mV at a frequency of 10 Hz. The electrolyte was 1 molar sodium perchlorate. This oxidation potential is in the range of oxidation potentials of many biologically important molecules and is close to the oxidation potential of gold surfaces on which many of the sensor devices are built. These examples describe the synthesis of two molecular species able to integrate into a self-assembled monolayer and including a moiety that promotes electron flow through a self-assembled monolayer, including a species having a redox potential less than 490 mV. Specifically, the electroactive species is a ferrocene including substituents selected to provide the ferrocene with a redox potential less than 490 mV. One ferrocene includes two methyl substituents, one on each cyclopentadiene ring (2), which oxidizes at 340 mV, and the other ferrocene includes eight methyl substituents, four on each cyclopentadiene ring (3), which oxidizes as 220 mV. That is, these species, 2 and 3, differ from 1 in the substitution of the ferrocene terminus of the molecule. The presence of eight and two methyl groups in 2 and 3 respectively serves to make the iron atom in the ferrocene unit more electron-rich and therefore lowers the oxidation potential (Connelly, N. G., Geiger, W. E; Chemical Reviews, 1996, 96, 877 and references therein) of compounds 2 and 3 relative to 1. The replacement of 1 with 2 or 3 now allows for the detection of the electric current from the ferrocene unit at low potential without interference from biologically occurring compounds. 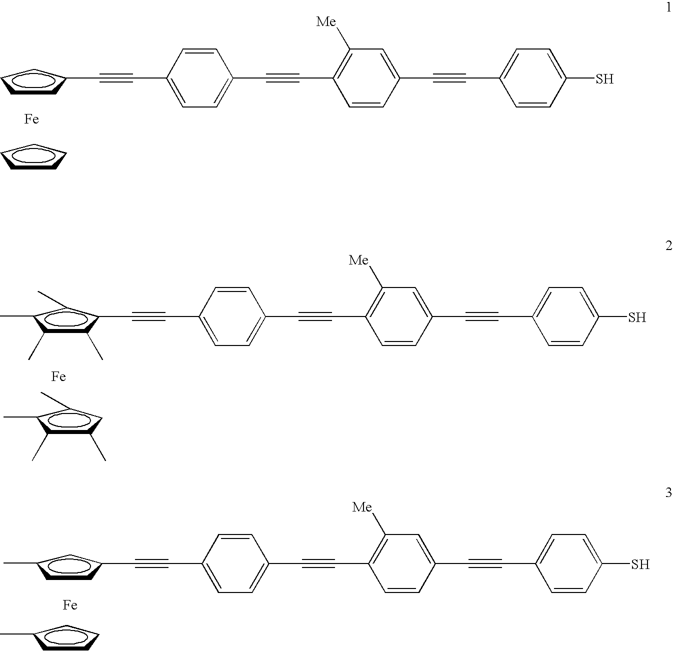
Preparation of Compounds 2 and 3
Synthesis 2 is outlined in Scheme 1. 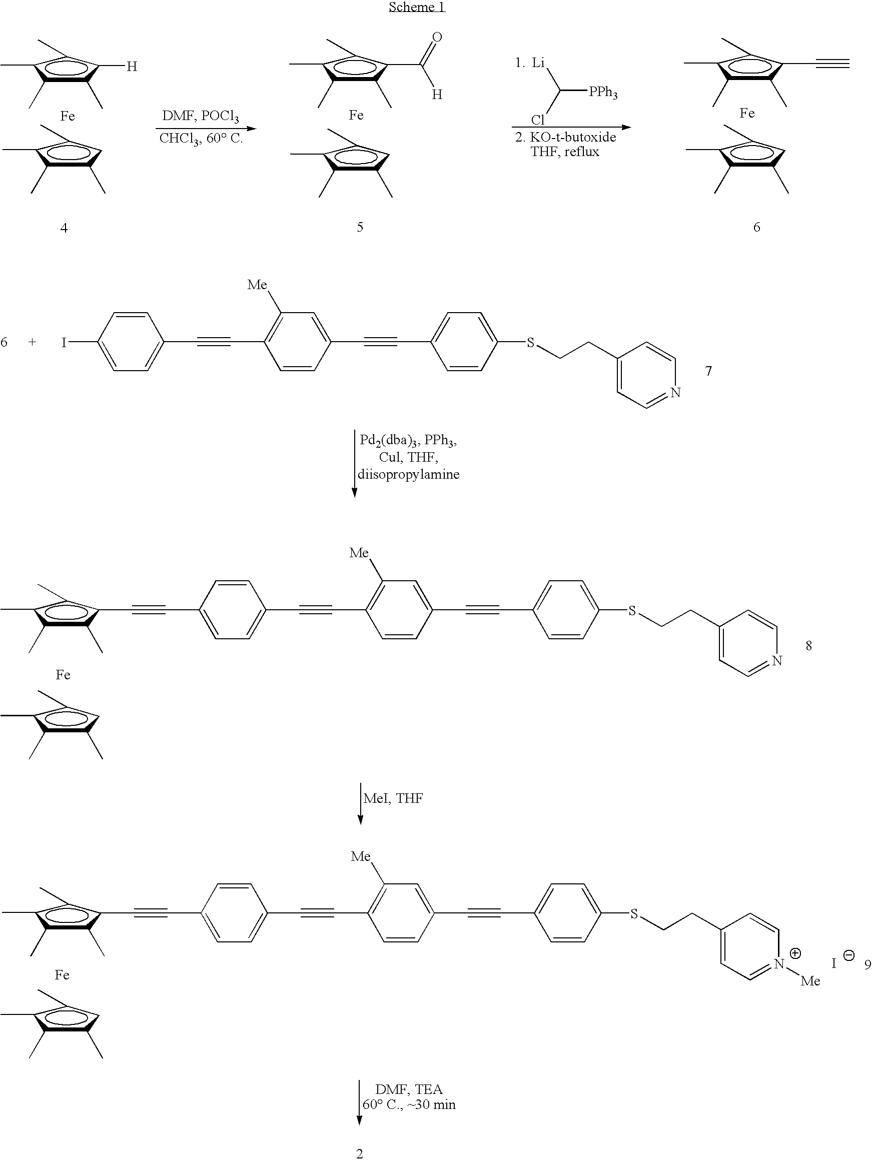
Synthesis of Compound 5.

Into a flame-dried sealable Schlenk flask with backflow of argon POCl₃ was added (0.26 ml, 2.8 equiv) and DMF (3 ml), followed by 4 (300 mg, 1 equiv) and DMF (1 ml). The resultant dark red reaction mixture was stirred at room temperature for 1 h and at 120° C. for 14 h. The reaction mixture was cooled in an ice/water bath, to it was added a solution of sodium acetate (1.508 g) in water (10 ml), and the mixture was stirred for 6 h. The mixture was extracted with ether (4×25 ml), the combined organic layers were washed with 1 M aqueous HCl (2×25 ml), saturated aqueous bicarbonate, and brine. The ether solution was dried with magnesium sulfate and concentrated. Attempts to purify the product by recrystallization from ether were not successful. The crude product was purified by flash chromatography on silica gel using 20:1 hexane:ethyl acetate as the eluant to obtain 42 mg of red solid.

Synthesis of 6.

To a suspension of (chloromethyl)triphenylphosphonium chloride (32 mg, 1.1 equiv) in THF (0.5 ml) was added n-butyl lithium (60 microL, 1.6 M in hexane, 1.1 equiv) dropwise, and the resultant solution was stirred for 20 min. A solution of 5 (27 mg, 1 equiv) in THF (0.5 ml) was added via cannula and the resultant red clear solution was stirred at room temperature for 3 h. Potassium t-butoxide was added, the reaction flask sealed, and heated at 70° C. for 92 h. The reaction mixture was diluted with water (10 ml), extracted with hexane (2×20 ml), dried with sodium sulfate, and concentrated. The product mixture contains the unreacted 5, the desired product 6, and the intermediate vinyl chloride 6a. The crude product was purified by flash chromatography on silica gel with hexane as the eluant to obtain 28 mg of an inseparable mixture of 6 and 6a in ratio of 1.8 to 1 as determined from the relative areas of alkynyl C—H proton to vinyl C—H in the product NMR spectrum. 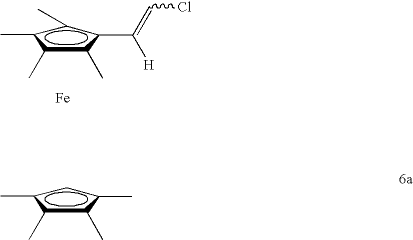
Synthesis of Compound 8

Into a flame-dried sealable schlenk flask under argon were loaded 6 (˜39 mg, 0.122 mmol, 1.25 equiv), 7 (54.4 mg, 0.098 mmol, 1 equiv), Pd₂(dba)₃ (4.5 mg, 0.05 equiv), PPh₃ (5.6 mg, 0.22 equiv), and CuI (2.8 mg, 0.15 equiv). Added THF (4 ml) and diisopropylamine (4 ml) and the resultant mixture was degassed thrice by application of pump vacuum followed by backfilling with argon. The flask was sealed and heated in oil bath with stirring at 55° C. for 48 h. The reaction mixture was diluted with dichloromethane and poured into aqueous ammonium hydroxide. The layers were separated, the aqueous layer was washed with additional dichloromethane, the organic extracts were combined, dried with sodium sulfate, and concentrated. The crude product was purified by flash chromatography on silica gel using 1:1 hexane:ethyl acetate as the eluant to obtain 56.1 mg of red solid.

Synthesis of Compound 9

To a solution of 8 (54 mg, 1 equiv) in THF (1 ml) under argon was added methyl iodide (40 microL, 10 equiv) and the reaction was stirred for 48 h. Volatiles were removed to obtain 62 mg of red semisolid.

Synthesis of Compound 2

A quantity of 9 is heated in a mixture of DMF and triethylamine to produce a solution of 2 that is used for further experiments.
Synthesis of 3 is outlined in Scheme 2. 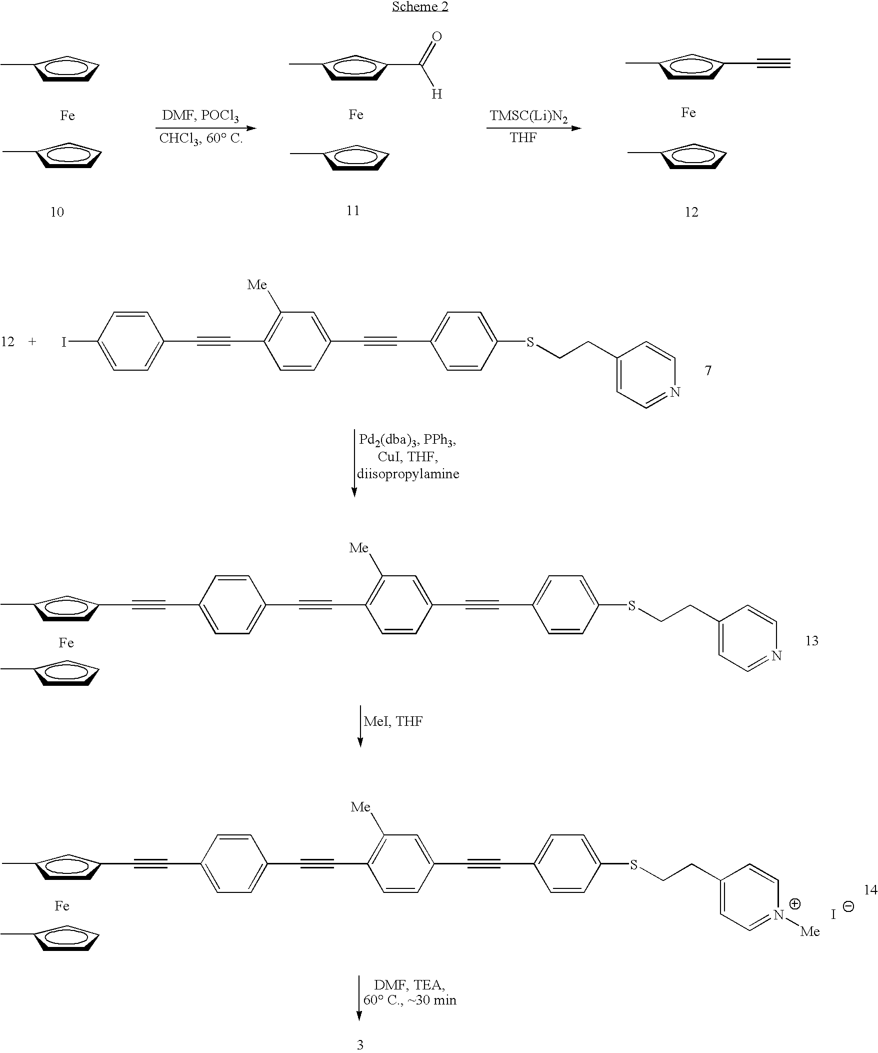
Synthesis of Compound 11

A solution of 1,1′-Dimethylferrocene 10 (Strem Chemicals, 0.5 g, 1 equiv) in chloroform (17.5 ml) containing DMF (1.05 ml, 5.8 equiv) and POCl₃ (2.86 g, 8 equiv) was heated at 50° C. with stirring under argon for 17 h. The solution was washed with saturated aqueous sodium acetate then 10% aqueous sodium bicarbonate, dried with magnesium sulfate, and concentrated. The crude product was purified by flash chromatography on silica gel with dichloromethane as the eluant to obtain 359.1 mg of 11.

Synthesis of Compound 12

n-BuLi (1.1 ml, 1.6 M in hexane, 1.2 equiv) dropwise to a solution of diisopropylamine (0.25 ml, 1.2 equiv) in THF (12 ml) at 0° C. The resultant solution was stirred for 1 h, cooled to −78° C. and to it was added trimethylsilyldiazomethane (0.89 ml, 2.0 M in hexane, 1.2 equiv), and stirring continued for 1 h. A solution of 11 in THF (6 ml) precooled to −78° C. was added via cannula, stirring continued for 1 h, and the reaction mixture was warmed to room temperature. The flask was sealed and heated at 65° C. for 72 h. The reaction mixture was poured into water, extracted with ether. The organic extract was dried with magnesium sulfate and concentrated. The crude product was purified by flash chromatography on silica gel using 4:1 hexane:ethyl acetate as the eluant to obtain 26.2 mg of 12.

Compound 13 was prepared from 12 and 7 by procedure identical to that used for the preparation of 8. Obtained 9.7 mg of 13.

Preparation of 14 is identical to that of 9.

Those skilled in the art would readily appreciate that all parameters listed herein are meant to be exemplary and that actual parameters will depend upon the specific application for which the methods and apparatus of the present invention are used. It is, therefore, to be understood that the foregoing embodiments are presented by way of example only and that, within the scope of the appended claims and equivalents thereto, the invention may be practiced otherwise than as specifically described. Specifically, those of ordinary skill in the art will recognize, or be able to ascertain using no more than routine experimentation, many equivalents to the specific embodiments of the invention described herein. Such equivalent are intended to be encompassed by the following claims.

In the claims, all transitional phrases such as “comprising”, “including”, “carrying”, “having”, “containing”, “involving”, and the like are to be understood to be open-ended, i.e. to mean including but not limited to. Only the transitional phrases “consisting of” and “consisting essentially of”, respectively, shall be closed or semi-closed transitional phrases as set forth in the United States Patent Office Manual of Patent Examining Procedures, section 2111.03.

1. A composition comprising:
a molecular species able to integrate into a self-assembled monolayer, comprising a metallocene including at least two substituents.

2-4. (canceled)

5. A composition as in claim 1, wherein the substituents comprise methyl groups.

6. A composition as in claim 5, wherein the metallocene is ferrocene.

7. A composition as in claim 1, wherein the metallocene is a ferrocene derivative.

8-13. (canceled)

14. A composition as in claim 1, the molecular species comprising a chemical group selected to adhere to a surface.

15. (canceled)

16. A composition as in claim 1, wherein the molecular species comprises an electroactive species having a redox potential less than 490 mV (vs Ag/AgCl).

17-32. (canceled)

33. A self-assembled monolayer comprising a plurality of molecular species as recited in claim 1.

34-36. (canceled)

37. A composition as in claim 1, wherein the molecular species comprises:
X—R—Y
wherein X comprises a functional group selected to adhere to a surface, R is a chemical bond, a spacer moiety that can form part of a self-assembled monolayer, a moiety that promotes electron flow through a self-assembled monolayer, or a combination, and Y comprises an electroactive signaling entity having a redox potential of less than 490 mV.

38-49. (canceled)

50. A kit comprising:
a first electroactive species having a first redox potential, immobilizable with respect to a first chemical or biological binding partner; and
a second electroactive species having a second redox potential, immobilizable with respect to a second chemical or biological binding partner.

51-55. (canceled)

56. A kit as in claim 50, wherein each of the first and second electroactive species comprises ferrocene including at least two substituents.

57-60. (canceled)

61. A kit as in claim 50, wherein the first electroactive species is immobilized with respect to the first chemical or biological binding partner and the second electroactive species is immobilized with respect to a second chemical or biological binding partner.

62. A method comprising:
determining a chemical or biological binding event indicated by a first redox potential of a first signaling entity in an assay involving also a second signaling entity having a second redox potential.

63. A method as in claim 62, wherein at least one of the first or second signaling entities comprises a metallocene.

64. (canceled)

65. A method as in claim 62, wherein each of the first and second signaling entities comprises ferrocene.

66-67. (canceled)

68. A method as in claim 62, comprising:
exposing an analyte to the first signaling entity, immobilized with respect to a first chemical or biological binding partner and the second signaling entity, immobilized with respect to a second chemical or biological binding partner;
allowing the first binding partner to bind to the analyte thereby immobilizing the first signaling entity with respect to the analyte; and
determining immobilization of the first binding partner with respect to the analyte.

69. A method as in claim 62, comprising exposing a first ligand immobilized with respect to a first signaling entity and second ligand immobilized with respect to a second signaling entity to a cell including a predetermined level of a first cell surface receptor that is a binding partner for the first ligand and a second cell surface receptor that is a binding partner to the second ligand, allowing the first ligand to bind to the first cell surface receptor and the second ligand to the second cell surface receptor, and determining the level of the second cell surface receptor at the cell surface from a signal from the second signaling entity calibrated with respect to a signal from the first signaling entity.

70-73. (canceled)

74. A method comprising as in claim 62, the first signaling entity comprising a metallocene the method comprising:
determining effectiveness of a candidate drug in inhibiting binding of a first binding partner to a second binding partner by exposing the first binding partner, linked to the metallocene, to the second binding partner in the presence of the candidate drug and determining binding of the first binding partner to the second binding partner by determination of the first redox potential of the metallocene.

75. A method as in claim 62, comprising:
determining effectiveness of a candidate drug in inhibiting binding of a first binding partner to a second binding partner by exposing the first binding partner, immobilized with respect to the first signaling entity, and the candidate drug, immobilized with respect to the second signaling entity, to the second binding partner and determining relative binding of the first binding partner or candidate drug to the second binding partner by determining proximity of the first or second signaling entity to the second binding partner.

76-77. (canceled)

78. A method as in claim 75, wherein each of the first and second signaling entities comprises a ferrocene.

79. A method as in claim 78, wherein the first signaling entity has a redox potential different from that of the second signaling entity.

80. (canceled)
